(19) United States
(12) Patent Application Publication
(10) Pub. No.: US 2011/0208222 A1
(43) Pub. Date: Aug. 25, 2011

(54) System and Method for the Treatment of Occluded Vessels

(76) Inventors: Boris Ljahnicky, Zagreb (HR); Gustavo Auerbach, Tel Aviv (IL)

(21) Appl. No.: 13/032,838

(22) Filed: Feb. 23, 2011

Related U.S. Application Data
(60) Provisional application No. 61/307,887, filed on Feb. 25, 2010.
(60) Provisional application No. 61/362,072, filed on Jul. 7, 2010.

(51) Int. Cl. A61B 17/22

(52) U.S. Cl. 606/159

(57) ABSTRACT

A system for treating an occlusive region of a blood vessel comprises: a catheter outer tube forming a lumen for inflation of a centering balloon; a catheter inner tube lined with either internal spiral threads or point slots to assist controlled guidewire movement; and a rotatable guidewire that may be solid or hollow. The guidewire comprises a rotatable body and a proximal shaft. The rotatable body has two components: a distal rotating head and a proximal rotating shaft, wherein these components are of equal length and the rotating shaft resides within the proximal shaft. Linear force applied to the proximal shaft will result in the rotational guidewire tip puncturing cutting through an occlusion. In an alternative embodiment, the guidewire rotatable body comprises two mechanical rings and a compression spring, wherein when the spring is released via depression of a button, the guidewire tip drills through an occlusion.

### PRIORITY CLAIMS TO RELATED PROVISIONAL APPLICATIONS

The present application claims priority benefit under 35 U.S.C. §119(e) to U.S. Provisional Patent Application Ser. No. 61/307,887, filed Feb. 25, 2010, entitled “System and Method for Treatment of Occluded Vessels”, and Ser. No. 61/362,072, filed Jul. 7, 2010, entitled “Occlusion System with Spring Activated Guidewire Rotation”. The present application incorporates the foregoing disclosures herein by reference.

### TECHNICAL FIELD

This invention relates generally to medical devices for treating vascular conditions. More specifically, the invention relates to a catheter incorporating a drilling guidewire.

### BACKGROUND OF THE INVENTION

Occlusions develop in a patient's vascular system, e.g., brain, heart, kidneys and peripheral arteries, when atherosclerotic plaque formations block or significantly reduce the flow of blood. Occlusions lasting three or more months are termed “chronic total occlusions” (CTO). Failure to remove the blockage can result in life threatening medical conditions, such as angina, hypertension, myocardial infarction, renal failure, and strokes.

Medical devices for CTO treatment have the ability to traverse through an occlusion in a patient's arteries for the purpose of repairing the lumen with balloon angioplasty and/or implantation of a stent. More often than not, this conventional protocol is unsuccessful because of the guidewire's inability to penetrate and traverse the occlusion. Traditional CTO devices have been subsequently augmented with additional devices which may comprise, for example, floppy motorized guidewires that generate high frequency reciprocal and lateral movements for the purpose of penetrating the proximal fibrous cap. This requires that the guidewire of the augmenting device be of sufficient mechanical and material strength to generate a focused force that the clinician may closely control within the artery. Often these guidewires are selected based upon their particular properties (i.e., stiffness, steerability, and difficulty in creating a subintimal pathway) and upon the step within a CTO procedure.

While most CTO devices have relied upon guidewires with straight distal ends comprising blunt or tapered tips, more recent devices incorporate the use of drilling tips that require the clinician to apply a manual or motorized torque force at the guidewire proximal end. This maneuver makes it difficult for the clinician to control the amount of force applied at the guidewire distal end, and hence the direction and distance the guidewire tip travels within an artery. Subsequently, there is an increased risk for damage to the vessel wall because of piercing or cutting by the guidewire tip. These rotational devices have also not demonstrated successful use with stiff guidewires because of their resistance in transmitting a torque force from the proximal to the distal end of the wire.

Additionally, while most CTO devices permit the occlusion debris to flow into the bloodstream, some devices provide for aspiration and/or filtering out of the debris to prevent any potential harmful effects from plaque material circulating in a patient's vasculature. These systems do not, though, provide for a mechanism to return the “cleaned” blood to a patient that was lost during the occlusion clearing and aspiration procedures, or to work within a rotatable drilling guidewire.

Therefore, there is a need within the medical industry for a guidewire that efficaciously and safely pierces and drills through an occlusion of all types (i.e., brain, heart, peripheral arteries, renal) and at all types of locations (e.g., bifurcations), with a device that requires minimal force to be applied by the clinician.

The prior art discloses a catheter encasing a rotating guidewire in U.S. Pat. No. 5,269,757 entitled “Catheter with Integrated Steerable Guidewire having Linear to Rotary Movement” discloses a complex mechanical device wherein the physician applies a linear force to a slider and hub attachment on the proximal end of the device. This will subsequently cause the guidewire with a helical winding at the midsection to traverse a part of the catheter with bent midsection which will cause the guidewire distal tip to rotate, although the tip is absent of a cutting tip. The present invention is an improvement over this device because of: 1) by only requiring an application of linear force to the proximal shaft (versus a slider), this invention is easier to use and provides more physician control in the amount of force applied and speed of guidewire rotation; 2) the catheter internal wall comprises helical windings that connect with and direct the helical windings of the guidewire, resulting in more rotations per linear distance and a faster rate of rotation; and 3) the guidewire of the present invention comprises a cutting and drilling tip versus a helical coil with a beaded tip (See U.S. Pat. No. 5,269,757, FIG. 1).

The prior art also discloses a device with a cutting tip and a guidewire tip for drilling through an occlusion. U.S. Patent Application 20100125253 entitled “Dual-tip Catheter System for Boring through Blocked Vascular Passages”, discloses a catheter encasing two separate devices: a guidewire wherein a linear force at its proximal end pushes the guidewire outside the catheter distal end; and a rotatable cutting tip wherein a linear force applied at its proximal end rotates the cutting tip to extend beyond the catheter distal end. The present invention is an improvement on this device by combining the rotating cutting tip with the guidewire under the directional control of a proximal shaft; therefore, resulting in a device which is easier for the physician to use by simultaneously cutting and drilling through an occlusion, and has more control over the distance and amount of force generated at the distal end.

### SUMMARY OF THE INVENTION

The present invention provides an occlusion treatment device that permits the clinician to apply a linear force on the proximal shaft to control a rotating guidewire tip's movement to within millimeters so as to effectively pierce a proximal fibrous cap. The device design enables treatment of all types of occlusions because of controlled maneuverability and safety within an artery. The occlusion treatment device also comprises a novel guidewire tip that has the ability to simultaneously cut and drill across a plaque formation. The system may also comprise a filtering system that simultaneously aspirates and filters out occlusion debris, and then returns clean blood to the patient, while providing an optional means to administer a therapeutic agent. This type of filtering system is especially efficacious in the use of arteries where there is a high occurrence of distal embolization, such as in the brain and kidneys.

The present invention is accomplished by providing a method and a system for a catheter drilling device comprising a catheter system with two tubes and a guidewire with a rotating head. The catheter outer tube forms a lumen for inflation of a balloon to center the guidewire within the blood vessel. The catheter inner tube possesses either internal spiral threads or point slots to assist the guidewire movement. And the guidewire distal end comprises a tapered head with spiral helical windings and a cutting tip (optional), and a rotatable shaft with two securing rings. The rotatable shaft is encased in a proximal shaft, wherein when linear force is applied to the proximal shaft, the distal tip of the guidewire rotates and advances to a predetermined length; preferably about 2 millimeters, beyond the catheter distal end, while puncturing an occlusion cap. This device is especially efficacious at bifurcations because of the ability of the inflated balloon to safely center the rotating guidewire against the proximal cap so as to prevent it from bending and traveling into the adjoining lumen.

The present invention may also comprise a filtering system within the guidewire of this invention, or any occlusion treatment device, that both aspirates the debrided occlusion, then filters and returns blood to the patient. The system comprises two parallel tubes with opposing flow directions, one for inflow during aspiration and one for outflow to the patient. The tubes are connected within a guidewire via a “Y”, “Reverse Y” or “H” shaped like configuration within which lie the placement of filters and valves with the tubes to remove debris and control the direction of flow.

The advantages of the present invention include, but are not limited to (i) providing the operator with the ability to rotate a stiff elongated body without using rotational forces; ii) minimizing the likelihood of vessel perforation; (iii) minimizing the potential that fragments of an occlusion can be carried to distal remote locations by blood flow, and (iv) providing a system suitable for occlusions considered to be of high risk of embolizations, such as renal arteries.

One aspect of the present invention is a system for treating chronic total occlusions, comprising a guidewire with a rotating drill tip and a centering catheter. The guidewire comprises a proximal shaft and a distal rotating body with a cone-like shaped tip, the two portions coupled, such that only rotational movements are enabled at the distal end when linear forces are applied at the proximal end.

Another aspect of the present invention is a system wherein the guidewire starts spinning and drilling through the proximal cap of the occlusion in rotational motion, while limiting its movement forward, and keeping the position of the tip centralized and away from the vessel walls.

Another aspect of the present invention is a catheter comprising two tubes, an outer tube and an inner tube forming a lumen for inflation of a centering balloon and an inner tube comprising spiral internal threads or point slots in the distal end that are arranged to enable passage of the guidewire through the catheter lumen.

Another aspect of the present invention is a centering balloon that secures the catheter in place within a vessel, and allows the catheter to direct the guidewire drilling tip towards the center of the proximal cap of the occlusion, therefore minimizing the risk of perforating the blood vessel or crossing into subintimal tissue.

Another aspect of the present invention is ability of the device to adapt to arteries of various diameters as compared to the device's, whereby the inflated balloon from the outer catheter secures the location of the guidewire within the artery.

Another aspect of the present invention is an enhanced ability to center the guidewire within the vessel, thereby improving the safety and performance of the device in treating CTO's. Centering is achieved via: contact between the inner catheter tube's rabbets and the guidewire windings; and via use of inflating a balloon from the catheter.

In a further embodiment of the present invention the guidewire is divided into a proximal shaft and a rotatable distal body, which may be secured within the shaft via two mechanical rings (distal and proximal). The proximal ring, or the proximal end of the rotatable distal body, are also attached to a compression spring mechanism. The guidewire tip rotates at high velocity and force upon release of the compression spring. The guidewire may also be used in conjunction with a conventional balloon catheter, and with the occlusion aspiration and filtration systems as disclosed herein.

Another aspect of the present invention is a filtering system with one-way valves within either the hollow guidewire of the present invention, or other catheter systems well known in the art. The filter system functions in conjunction with the hollow guidewire to simultaneously drill and aspirate debris and then return the filtered blood to the body. Use of the filtering system with the disclosed guidewire/catheter system allows treatment of all types of occlusions, to include those located within the brain and kidneys because of the filter's ability to prevent life threatening embolizations.

Another aspect of the present invention is that a centering catheter or guidewire may comprise a radiopaque marker or other imaging modalities so as to improve the success of crossing of the lesion, while minimizing the risk of perforating the blood vessel or crossing into arterial subintimal tissue.

### BRIEF DESCRIPTION OF THE DRAWINGS

FIG. 1 is a perspective view of the catheter system to include the outer and inner catheter tube, the guidewire, and the balloon inflated.

FIG. 2A is a side view of the guidewire distal body and the inner catheter tube with threads.

FIG. 2B is a side view of the guidewire distal body with one winding and the inner catheter tube with threads.

FIG. 2C is a side view of the guidewire distal body and the inner catheter tube with point slots.

FIG. 3A is a perspective view of the guidewire distal end.

FIG. 3B is a perspective view of the guidewire distal end with a cutting tip added.

FIG. 4A-E show different embodiments of coupling between rotatable body and guidewire proximal shaft.

FIGS. 5A and 5B illustrate the catheter system with the inflation lumen (5A) and the inflation tube (5B).

FIG. 6 is side view of the “Reverse Y” filtration system.

FIGS. 7A-C are cutaway views of different embodiments of the “Reverse Y”

filtration system: inflow and outflow tubes in tangent (A); tubes separated (B); tubes connected with perforated wall (C)

FIG. 8 is a cutaway view of the “H” filtration system with inflow and outflow tubes.

FIG. 9 is a cutaway view of the “Y” filtration system with inflow and outflow tubes.

FIGS. 10A and 10B are top views of the one way flow valves in closed and open positions.

FIGS. 11A-D are side views of different embodiments of coupling between the guidewire rotatable body and guidewire proximal shaft and attachment of the compression spring.

FIG. 12 is a side view of the guidewire rotatable body with two rings and a compressed spring, with a metal extensor inserted into a slot to prevent guidewire rotation.

### DETAILED DESCRIPTION

In the present invention the term “occlusion” refers to partial and total occlusion located within any artery. The present invention comprises a centering catheter with two tubes, one within another, and a guidewire with a rotating distal end. The guidewire may be solid or hollow. For example, in an alternative embodiment, the guidewire is hollow and houses a filtration system to aspirate debris and return clean blood to the occlusion site. The guidewire comprises a rotatable body and a proximal shaft. The rotatable body has two components: a distal rotating head and a proximal rotating shaft, wherein these components are of equal length and the rotating shaft resides within the proximal shaft.

Guidewires are used by medical clinicians for a variety of invasive procedures, and thus vary in size and stiffness in accordance with the type of procedure. The guidewire of the present invention may be about 120 centimeters to about 300 centimeters long, with a length of about 145 centimeters often being used. The outer diameter of the guidewire may range from about 0.010 inches to 0.038 inches, and preferably is about 0.014 inches. However, it can have other sizes readily apparent to the skilled artisan. The guidewire may also have varying degrees of stiffness based upon the procedure. Softer, floppier wires are less likely to perforate vessel walls and are therefore deployed for navigating through tortuous arteries. But, more rigid guidewires are required to penetrate and cross occlusions while risking puncturing or tearing lumen walls. An advantage of the present invention is that the operator is not forced to compromise rigidity for a reduced risk of lumen puncturing because the rapid rotation of the guidewire head creates additional force sufficient to penetrate an occlusion.

### Centering Catheter

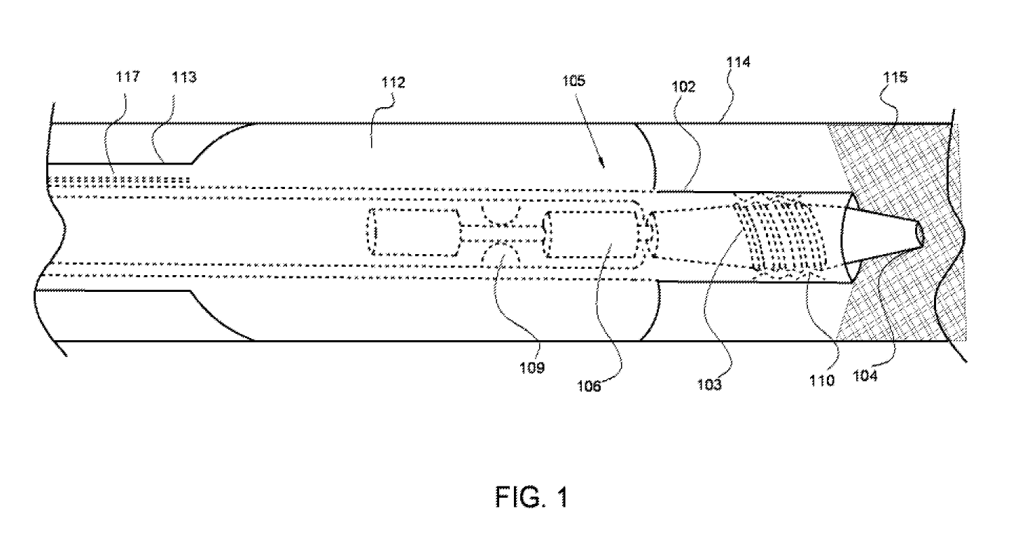

As shown in FIG. 1, the outer catheter tube 113 forms a lumen 118 for inflation of a balloon 112. The catheter inner tube 102 permits passage of the guidewire distal body 105 in a rotating motion. During insertion of the guidewire into the inner catheter tube, the guidewire's helical windings 103 follow the path of the catheter's rabbets 110, similar to a screw, while keeping the guidewire centralized. By applying a linear force at the guidewire proximal end to advance the guidewire through the catheter, a rotational movement is generated in the guidewire's distal end. The rotation stops when the drilling tip extends out of the distal end of the catheter to a predetermined length of 1-3 millimeter, and preferably 2 millimeter. Thus, pushing forward of the guidewire through the top of the catheter enables a linear force being converted to a torque force, without external manual or motorized rotational force being applied to the system.

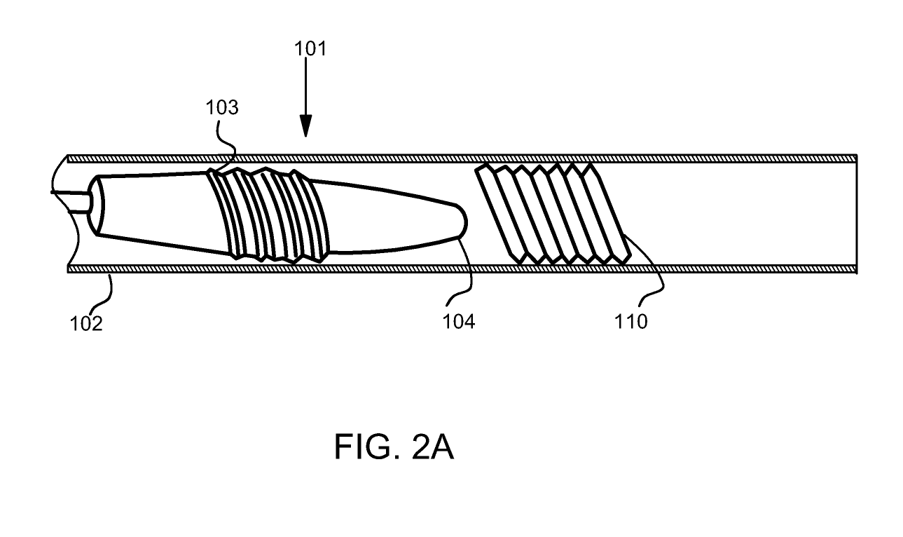

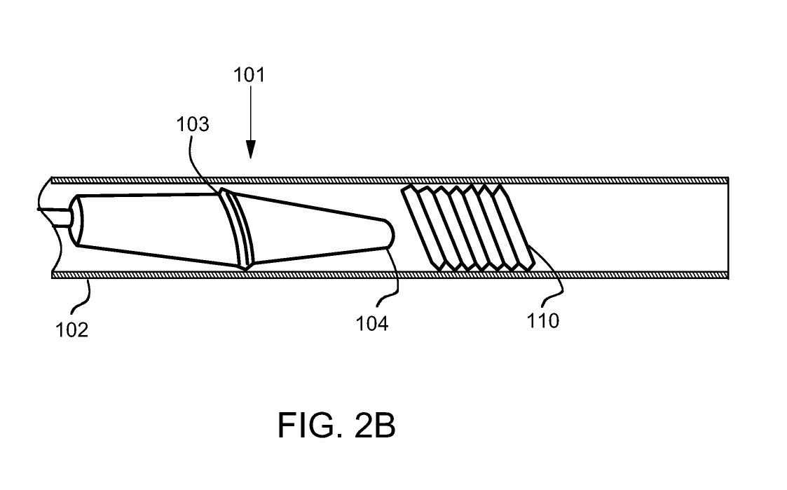

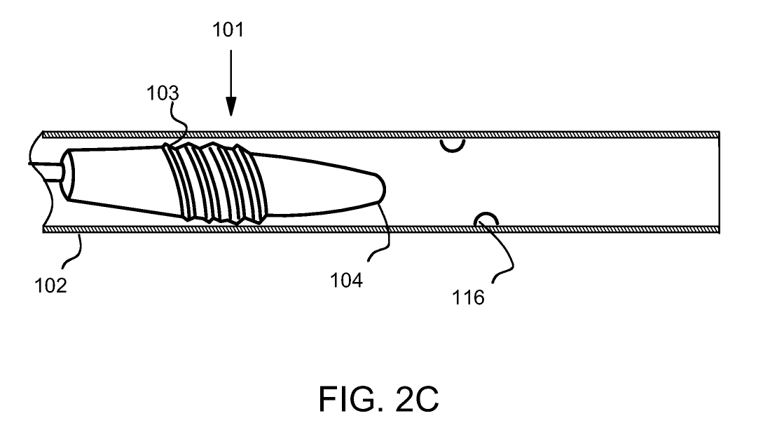

Inner Catheter Tube Embodiments: As illustrated in FIGS. 2A-C, there are multiple embodiments of the inner catheter tube. The catheter/guidewire system of FIG. 2A is a “screw-like” device, wherein spiral threads 110 lining the inner catheter tube 102 distal end slide along the rabbets lying between the guidewire tip's helical windings 103. Applying a translational force to the proximal shaft of the guidewire causes its rotatable distal body 105 to spin and advance through the lumen of the inner catheter tube 102. Rotation movement stops when all the guidewire winding(s) finish passing across all the catheter threads. Therefore, the length extension of guidewire tip beyond the catheters can be controlled by the disposition of the internal rabbets or point slots. The last point contact of a rabbet-winding at the device distal end is what determines the extension length of the tip, which could be 1-3 mm, and preferably 2 mm.

There are various possible combinations in the angle and number of spiral internal threads 110 or point slots 116 and guidewire helical windings 103, which dictate the length the guidewire will extend beyond the catheter. The angle of the guidewire windings 103 and the angle of the inner catheter threads 110 can be altered within a range from 30 to 45 degrees. FIG. 2B shows a specific embodiment where the inner catheter tube 102 has multiple spiral internal threads 110 corresponding with a single helical winding 103 at the rotatable distal body 105 of the guidewire. In different embodiments, the same catheter 102 with multiple spiral internal threads 110 may correspond with a guidewire having two or more helical windings 103. In other different embodiments, a catheter 102 may have one or two spiral internal threads 110 corresponding with one, two or multiple helical windings 103 at the rotatable body 105 of the guidewire.

In another embodiment as shown in FIG. 2C, the catheter 102 has two point slots 116, which are pointed, elevated structures emerging from the lining of the inner catheter tube 102. Applying a translational force to the proximal of the guidewire causes its cone shaped tip 104 to pass the point slots unhindered. But, when the guidewire rabbets, which lie between the windings 103, connect with the slots 116, the guidewire distal body 105 will spin and advance through the lumen of the inner catheter tube 102. Rotation movement stops when all the guidewire winding(s) 103 finish passing across all the point slots 116. Similarly to the embodiments described above, the catheter 102 having point slots 116 may work with any number of helical windings 103 on the guidewire.

### Rotating Guidewire

The guidewire may be about 120 centimeters to about 300 centimeters long, with a length of about 145 centimeters often being used. The outer diameter of the guidewire may range from about 0.010 inches to 0.038 inches, and preferably is about 0.014 inches. However, it can have other sizes readily apparent to the skilled artisan. In order to accept other angioplasty devices without interference of internal catheter structures (rabbets, point slots), the inner diameter should be slightly higher than “normal”, and all dimension should take into account both catheter and guide wire structures (rabbets, windings), while keeping functionality of conventional angioplasty devices that will be used later after successfully crossing the proximal cap. The inner catheter tube is preferably sized to accommodate guidewires of about 0.014 inch, but may be dimensioned to work with larger or smaller diameter guidewires, and accept other conventional angioplasty devices. An outer diameter of inner catheter tube is preferably about 0.020 inches to about 0.060 inches, and most preferably is about 0.021 inches to about 0.040 inches. Two radiopaque bands on the distal end allow its proper positioning under fluoroscopy.

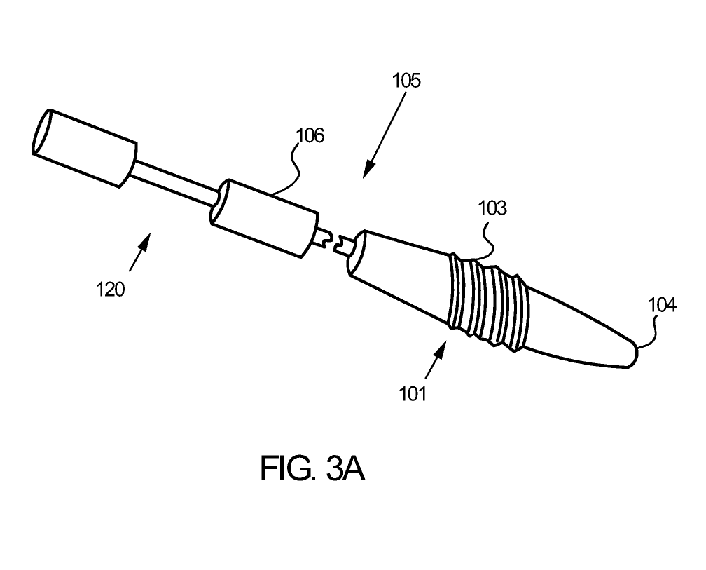

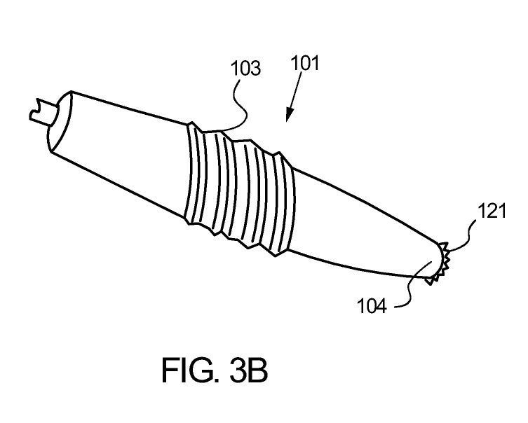

Another feature of the present invention is the stiffness and composition of the guidewire are not primary factors in the ability of the device to effectively clear a CTO, unlike other CTO devices. The mechanical and material properties of the guidewire of the present invention are augmented or superseded by the device's novel shaft rotation design that controls the direction and increases the amount of force applied at the guidewire tip. The guidewire of the present invention is sufficiently strong to pierce the proximal fibrous calcified cap. At which point another guidewire of less stiffness is exchanged through catheter 102 to traverse the lesion. Alternatively, a variety of cutting tips 121 may be added to the end of the guidewire of the present invention in order to drill through an occlusion in a manner similar to an artherectomy device (See FIG. 3B).

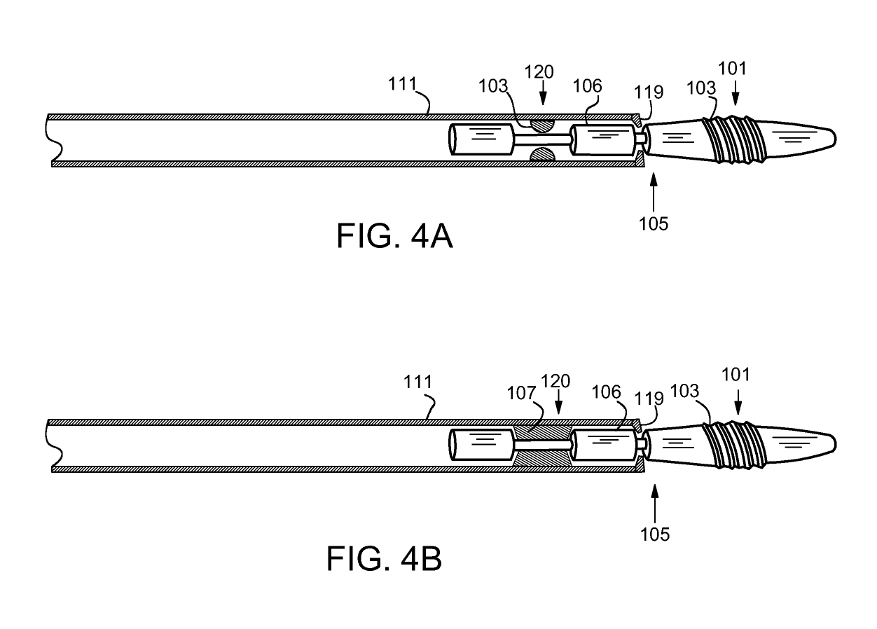

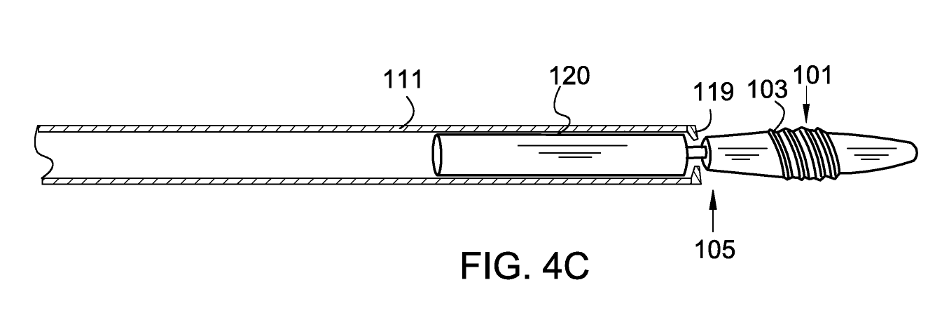

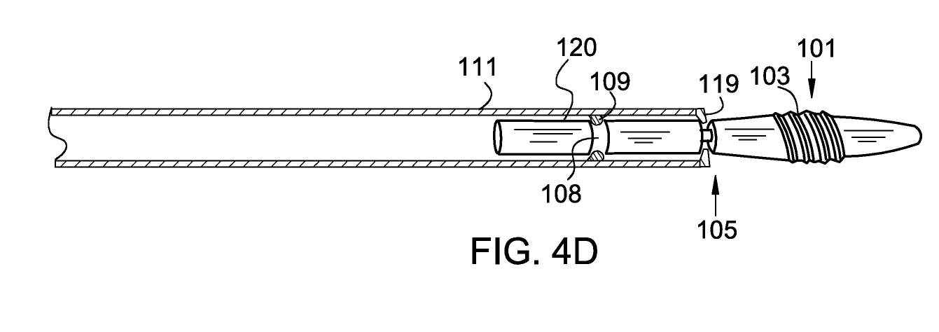

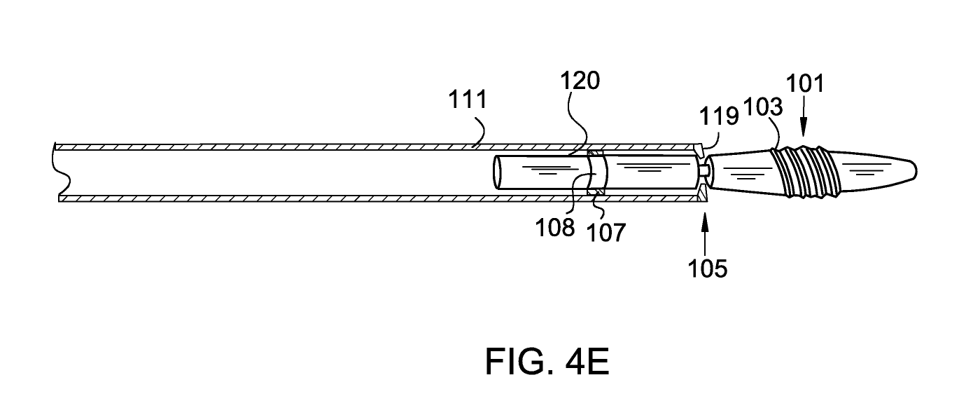

FIGS. 3A, 3B, and 4A illustrate the guidewire, which comprises a proximal shaft (111) encasing the proximal half of a distal rotatable body (105). The rotatable body 105 comprises two components (120, 101), which are equivalent in length in the absence of a filtration system. The distal portion of the rotatable body 105 comprises a tapered head 101 with windings 103, and alternatively, a cutting tip 121. The rotational shaft 120 is encased by the proximal shaft 111, so that rotatable body 105 remains stable without trembling and side movement while spinning. Rotational shaft 120 further comprises at least one, preferably two securing rings 106. The ring(s) 106 function to secure the distal body 105 into the guidewire proximal shaft 111 at a point about 0.5 mm below the narrowing at 119 (FIG. 4A).

One or more helical windings 103 are wrapped around the rotatable body 105, having preferably an angle disposition of 30°-45°, to aid in the passage of the rotatable distal body 105 through the distal end of the inner catheter tube 102. The speed of rotational movement varies according to the angle orientation of the helical windings 103. An angle of 30°-45° was found to be optimal for easy passage of the rotatable body 105 into the end of the inner catheter tube 102, while providing enough torque force to cross the calcified occlusion cap. (Blunter angles make the rotation too slow to penetrate the proximal calcified cap of the vessel occlusion, and sharper angles increase the rotational speed too quickly to maintain proper control of the guidewire.)

FIGS. 4A-E show different embodiments of coupling between rotatable body 105 and guidewire proximal shaft 111. The guidewire proximal shaft 111 has a narrowing distal end 119, such that it prevents the rotatable shaft 120 from becoming detached from the guidewire proximal shaft 111. In a preferred embodiment shown in FIG. 4A, the rotatable shaft 120 encased by the guidewire proximal shaft 111 is about one third smaller in diameter than the distal part of the rotatable body 105 entering into the catheter 102. Two rings 106 are secured around the rotatable shaft 120, the distal ring by clockwise winding, so that a clockwise rotation movement enables further screwing and security clockwise, and proximal ring by counterclockwise winding, so that a counterclockwise rotation movement enables further screwing and security counterclockwise, such that the rings 106 will not be disconnected from the rotatable shaft 120. Two indented slots 109 emerging from the inner catheter wall are disposed between the rings 106 diagonally on the guidewire body diameter, securing the rotatable body 105 to guidewire proximal shaft 111, preventing movements distally, proximally and laterally, while enabling free rotational movement.

In another embodiment shown in FIG. 4B, an elevated circular slot 107 is disposed close to the rotational shaft 120 between the rings 106 securing the rotatable body 105 to guidewire proximal shaft 111, preventing movements distally, proximally and laterally, while enabling free rotational movement.

In another embodiment shown in FIG. 4C, the guidewire proximal shaft 111 has a narrowing distal end 119, such that it prevents the rotatable shaft 120 to be detached from the guidewire proximal shaft 111. The rotatable shaft 120 has just about the diameter as the guidewire proximal shaft 111, such that it nearly contacts the inner wall of guidewire proximal shaft 111, allowing rotation while preventing side movements.

In another embodiment shown in FIG. 4D, the rotatable shaft 120 encased by the guidewire proximal shaft 111 is smaller in diameter than the distal part entering into the catheter 102. The rotatable shaft 120 has a circular channel 108 around its diameter. The guidewire body may have two indented slots 109 emerging from the guidewire body inner wall and disposed diagonally on the body diameter. The indented slots 109 allow coupling with the circular channel 108 of the guidewire proximal shaft 111, securing the rotatable body 105 to guidewire body from moving distally or proximally and preventing side leaning, while enabling free rotational movement.

In another embodiment shown in FIG. 4E, a circular slot 107 emerging from the guidewire body inner wall is coupled with the circular channel 108 of the guidewire proximal shaft 111, securing the rotatable body 105 to guidewire body from moving distally or proximally and preventing side leaning, while enabling free rotational movement.

### Balloon Centering—Inflation Lumen & Inflation Tube

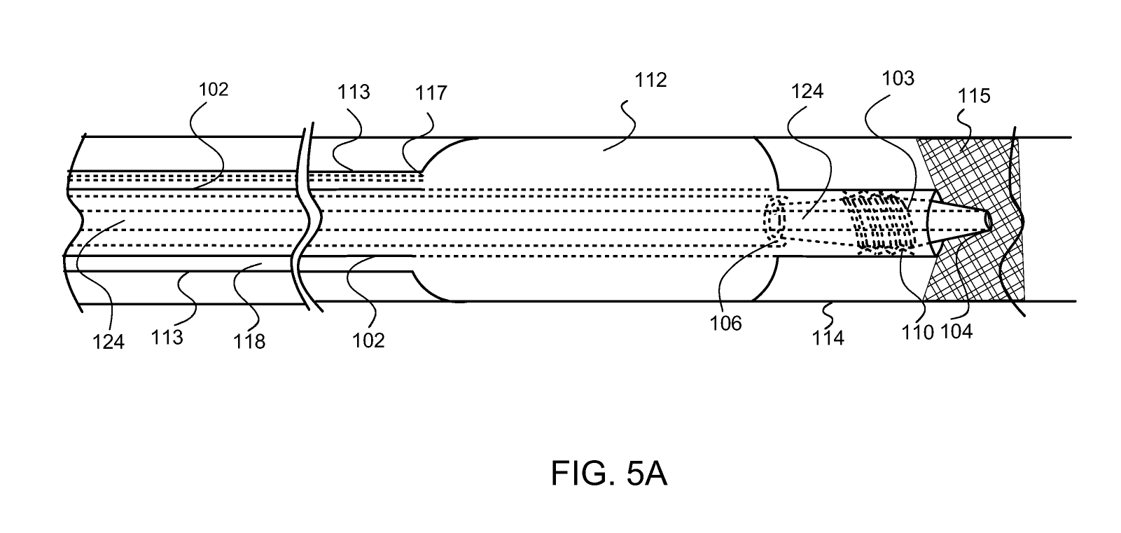

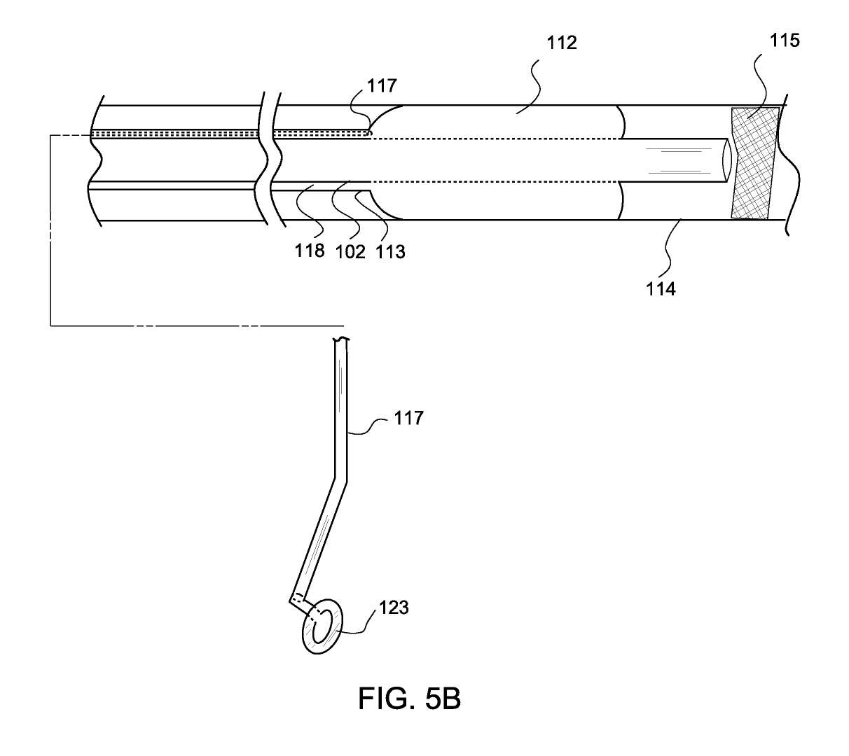

As illustrated in FIGS. 1 and 5A, another aspect of the present invention is a system to inflate a balloon 112 from the outer catheter tube 113, preferably at the site of the occlusion 115 within an artery. The inflated balloon 112 will centralize and secure the inner catheter tube 102 and guidewire drilling tip 104 relative to the vessel wall 114, especially when the diameter of the artery is larger than the catheter. It will also secure the guidewire of the present invention at the center of the proximal cap and occlusion body 115, thus minimizing the risk of perforating the blood vessel 114 or crossing into subintimal tissue. It is also noted, the device is centralized not only by the balloon 112, but also by the contact between the guidewire windings 103 and the inner catheter tube threads 110. As illustrated in FIG. 5A, the catheter system is positioned near the occlusion 115. The balloon 112 is inflated, securing the balloon 112 against the vessel wall 114 and ensuring that the distal end of the inner tube 102 is centered relative to the vessel wall 114. Once fully inflated the balloon 112 will extend about 0.5 cm from the distal end of the catheter to secure and center the system into place, while not making contact with the guidewire drill tip 104. When the guidewire is inserted and goes through the lumen of the inner tube 102, the drilling distal tip 104 of the guidewire 100 is directed towards the center of the proximal end of the calcified cap 115. By keeping the inner tube 102 centered in the vessel, the guidewire is directed to maintain a true lumen position, and away from the vessel wall 114.

In another embodiment, as illustrated in FIG. 5B, an inflation tube 117 extends across the passageway lumen 118 formed between the outer catheter tube 113 and inner catheter tube 102. A circular plastic button 123 has a diameter such that it fits a standard syringe and it is placed at the proximal end of the inflation tube 117, where it controls the opening and closing of the inflation tube 117, thereby inflating or deflating the balloon 112, as desired by the operator. The balloon 112 is inflated by introducing sterile liquid or gas (air) through a syringe into the passageway lumen 118 or inflation tube 117. The passageway lumen 118 or inflation tube 117 is opened by rotating the button 123 in a 90° clockwise direction, enabling the liquid or gas to go through, and is then closed by rotating the button 123 in a 90° counterclockwise direction as shown in, thus preventing the loss of liquid or air and ensuring dilatation of the balloon 112.

An inflatable balloon 112 extends distally at a distance of 0.5 cm from the distal end of the central tube 102, such that the rotatable distal portion 105 of the guidewire does not contact with the balloon 112. A syringe (not shown) is used to inflate the balloon 112 by delivering a predetermined volume of liquid in a regulated, low pressure manner, preferably to a pressure of 1.5 atm, such that it will not cause leaks and also to allow the guidewire to freely pass through the catheter 102. Applying higher inflation pressure may result in gripping of the guidewire and axial elongation of the balloon 112. A small amount of liquid is applied to narrow arteries, preferably about 0.5 to 2.5 milliliters, whereas a volume as big as 5 milliliters may be given when wider lumens are involved. When the balloon 112 is inflated, the distal portion of the balloon 112 is not elongated.

Bifurcation and Tortuous Small Arteries. A CTO may occur at the site of a bifurcation, wherein the blockage occurs within one blood vessel at a point just beyond where a main vessel branches into two smaller vessels. These are particularly difficult CTO's to treat because the guidewire will often bend upon contact with the occlusion and travel down the opposite branch. When the present invention is used to treat a CTO at the site of a bifurcation, the centering balloon is inflated at a point distal to the bifurcation and flush with the occlusion. The guidewire is then advanced through the center point of the proximal cap without the risk of it puncturing the vessel wall or traveling into the adjoining branch. Similarly, in tortuous small arteries, the device is placed flush with the proximal cap, and the guidewire is of sufficient stiffness that it crosses through the CTO.

Rotational Guidewire with a Filtration System

The present invention further includes the combination of a filtering system situated inside the rotating guidewire, or with any combination of conventional guidewires or catheters. The filtration system comprises two parallel tubes with opposing flow directions, one for inflow during aspiration and one for outflow of clean blood back to the patient. The tubes are connected within a guidewire via a “Y” (negative pressure system), a “Reverse Y” (positive pressure system), or an “H” like configuration. Within each configuration lie a unique combination of filters, narrow tubes, and one way flow valves for directing the removal of debris generated from the cutting and drilling of the occlusion, returning filtered blood to the patient, and optionally administering therapeutic agents or contrast imaging agents to the occlusion site.

Positive Pressure System with Saline Injection

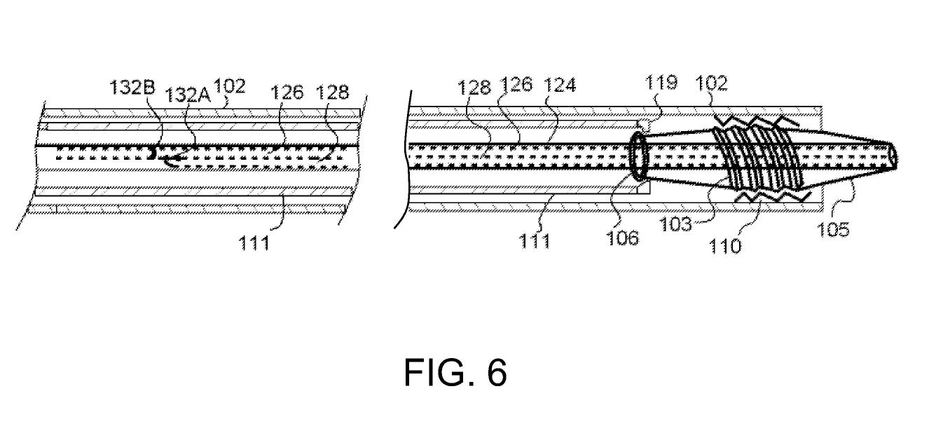

As shown in FIG. 6, the guidewire distal body 105 is rotating around a centrally placed tube 124, through which two smaller interconnected tubes housing the filtration system are located (126, 128). The central placed tube 124 serves as a shaft, wherein the only movable part of the guidewire is the rotating distal body 105. The proximal part of the distal body 105 is smaller in diameter than the guidewire proximal shaft 111 at their point of attachment. The proximal shaft 111 has a narrowing distal end 119, wherein a ring or nut 106 further secures the distal body 105 into the guidewire proximal shaft 111 at a point about 0.5 mm below the narrowing at 119. This connection prevents detachment of the distal body 105, while ensuring stable rotation without side movements and trembling. It is noted that when the present guidewire invention is used with the filtration system, then only one ring 106 is required to connect the two bodies, whereas two rings 106 are required when there is not filtration system. Alternatively, the connection point may contain two rings 106 to further stabilize the movable part as described in other embodiments. Additionally, when a filtration system is used, there is no requirement for equivalent lengths between the proximal rotational shaft 120 and the rotatable distal body 101.

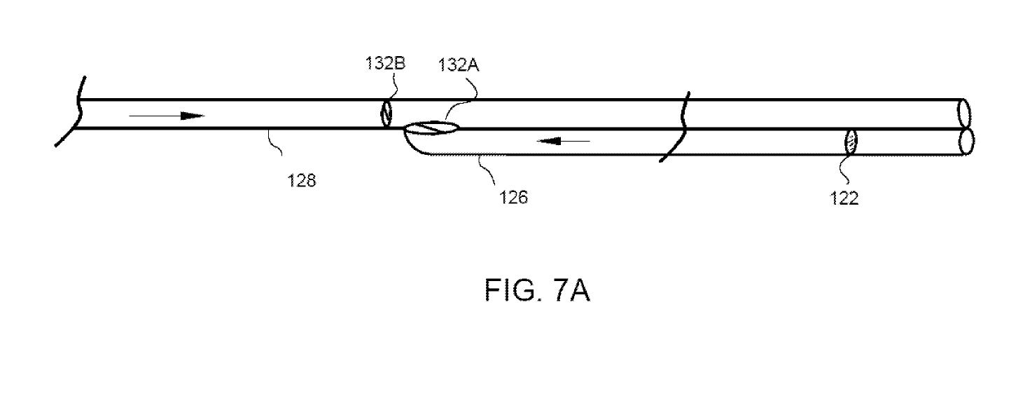

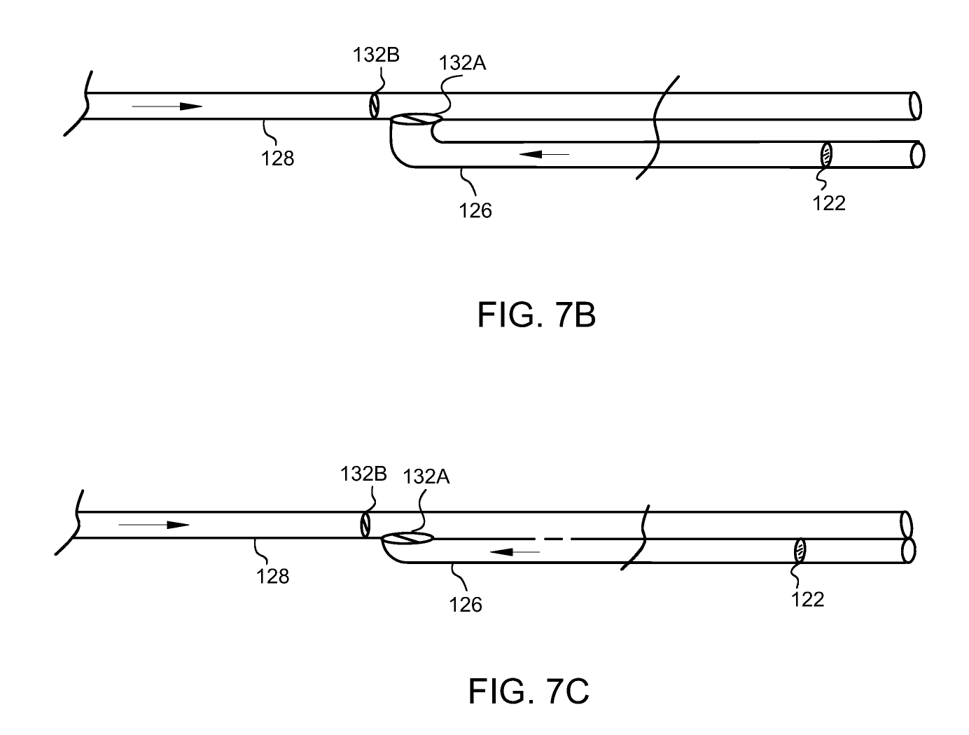

FIG. 6 illustrates the “Reverse Y” embodiment of the catheter system with the filtration system. The central tube 124 runs the entire length of the guidewire, and serves as a shaft for the guidewire distal body 105 rotation. As illustrated in further detail in FIG. 7A. there is a tube system within the central tube 124 (FIG. 6), for filtering the blood of debris and dust created during drilling and crossing of calcified plaque, comprising two interconnected tubes. The left inflow tube 128 is the same length as the central tube, and thus the guidewire. The right outflow tube 126 is approximately 8-10 cm long. (Outflow and inflow refer to the direction of blood upon leaving and entering the patient's body.) A filter 122 is located approximately 3-5 cm from the distal end of the right tube, for collecting the dust and debris created during drilling. In another embodiment as illustrated in FIG. 7B, the inflow and outflow tube may be slightly separated. Alternatively, there can be two filters 122, a distal filter and a small proximal filter, located about 2 cm apart. Occlusion filters that are well known in the art may be used with the present invention.

Prior to the drilling of the occlusion site, a saline solution is injected and squirted into the left tube 128 via a syringe, such as a 20 milliliter syringe. The squirting of saline solution through the left tube 128 enables the following: creating negative pressure in the right tube 126 to flush debris caused by drilling of calcified plaque; and preventing entrance of air from the left tube 128 into the occlusion site and thus the creation of an air embolism. Additionally, the left tube 128 can be used to administer additional solutions such as contrast solution, heparin, and other therapeutic or diagnostic agents at doses well known in the art.

The filtration system has at least one, and preferably two one way flow valves, as shown in FIGS. 7A and 7B. Valve 132A sits at the connection point between the right outflow and left inflow tube. Valve 132B resides within the left inflow tube 128 and below the connection point. When valve 132A is open, it generates continuous flow from the right outflow tube 126 to the left inflow tube 128. Additionally, the saline injection is continuous and produces sufficient fluid velocity in the left inflow tube 128 to generate positive pressure in the tube and concurrent negative pressure in the right outflow tube 126. The negative pressure is of sufficient force to aspirate blood containing dust and debris created during the guidewire tip's drilling of an occlusion. Other valves that are well known in the art for having demonstrated the ability to firmly block fluid backflow are suitable for use with the present invention. The filter 122 disposed along the right outflow tube 126 traps particles of dust and debris while allowing blood to go through. Valve 132B prevents filtered blood from flowing into the proximal end of the guidewire, while valve 132A allows the filtered blood to be returned to the patient via the left inflow tube 128.

In another embodiment of the present filtration invention, as illustrated in FIG. 7C, the two tubes have a common inner wall which has several perforations connecting between them. The flow of saline solution through the left inflow tube 128 creates a negative pressure in the right outflow tube 126. Part of the saline solution enters into the right outflow tube 126 through these perforations, and part of the aspirated blood can enter into the left inflow tube 128 with positive pressure. The mixing of liquids does not cause any disturbances in the system or complications.

### Negative Pressure Filtration System

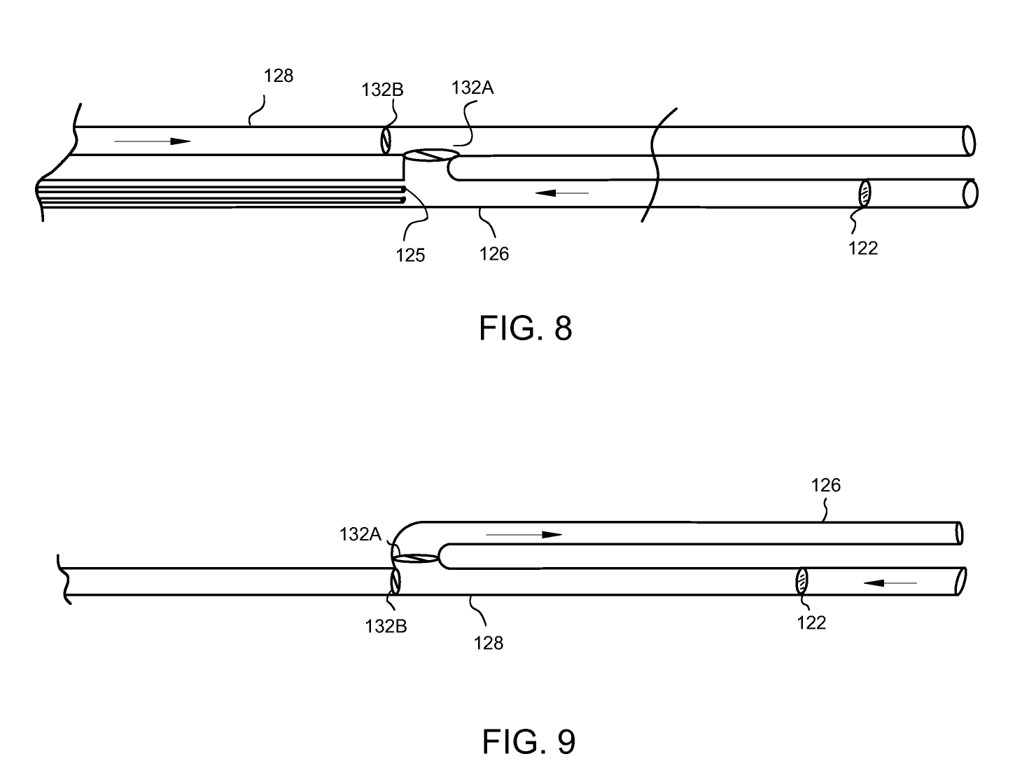

In a further embodiment of the present invention, the “H” filtration system as illustrated in FIG. 8, the right tube is connected to a vacuum apparatus at the proximal end of the guidewire, which creates a negative pressure to aspirate the occlusion site. At the connection point between the inflow and outflow tubes, very thin, narrow (micro) tubes lying in parallel extend from (125) to the proximal end of the guidewire, where they merge with the vacuum source. The presence of several narrow tubes 125 causes resistance to aspirated blood flow, thus forcing the filtered blood to continue flowing into the left tube 128 with positive pressure while preventing the aspirated blood to flow through the right tube 126 with negative pressure towards the vacuum source. By creating both negative and positive pressure, only a small amount of the aspirated blood may end up in the vacuum source, and be collected in the vacuum tank. Additionally, the left tube 128 can be used to administer additional solutions such as contrast solution, heparin, and other therapeutic or diagnostic agents at doses well known in the art.

An alternative negative pressure filtration system replaces the narrow parallel tubes with a one way flow valve. As illustrated in FIG. 9, the “Y” shaped like filter system comprises a central tube or lumen 124 of a guidewire, which encases an inflow (positive pressure) tube 126 and an outflow (negative pressure) tube 128 lying in parallel. The inflow and outflow tubes (126, 128) emerge from the distal opening of the guidewire tip; and on the proximal end, the two tubes merge into a single tube which is connected to a vacuum source. In a specific embodiment, outflow tube 128 is positioned slightly laterally with respect to the guidewire distal tip so as to facilitate blood aspiration. The filter system further comprises at least one filter element 122, and preferably two filter elements, cross-sectioning the outflow tube 128 so as to retain the aspirant debris from the occlusion. Occlusion filter devices well known in the art may be used for element 122. And as shown in FIG. 9, the filtration system further comprises at least two one way valve members (132A, 132B) to re-direct the flow of filtered blood back to the patient.

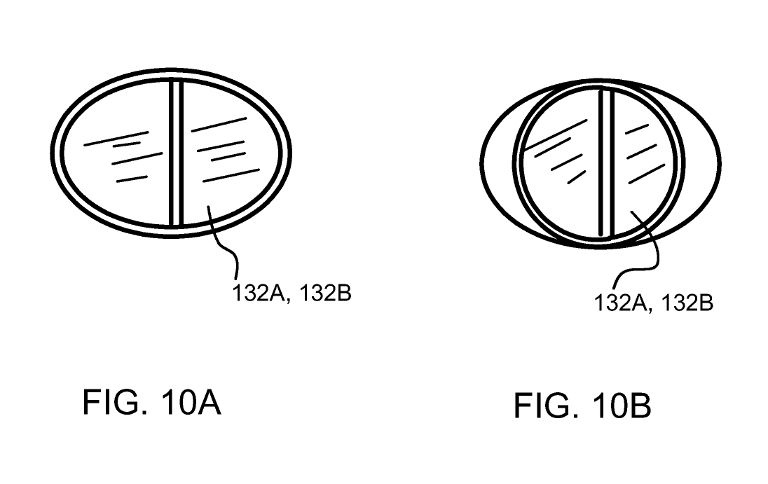

Valve Design: The present invention comprises several embodiments of one way flow valves 132A and 132B. the valve members of an outflow valve 132A may include two or three leaflets, each composed of a flexible material capable of deflecting to the open position, as illustrated in FIG. 10B, when under fluid or air pressure, and then returning to the closed position, as illustrated in FIG. 10B, when the pressure falls. Preferably, the material will be rubber. Other valves that are well known in the art for having demonstrated the ability to firmly block fluid backflow are suitable for use with the present invention.

### Spring Activated Guidewire Rotation

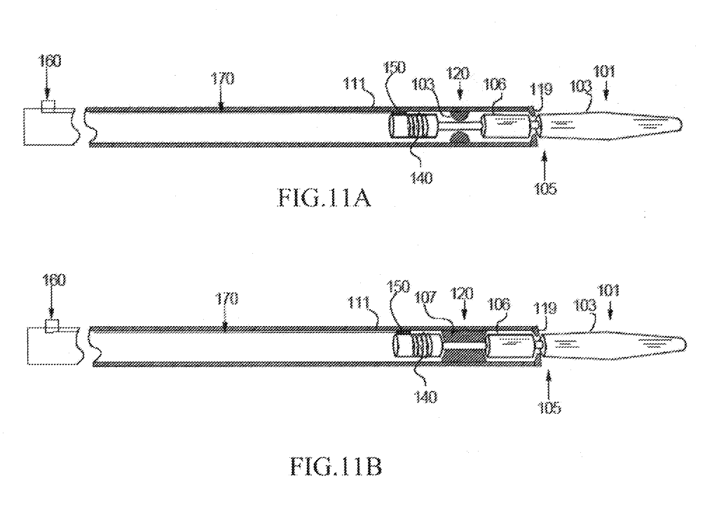

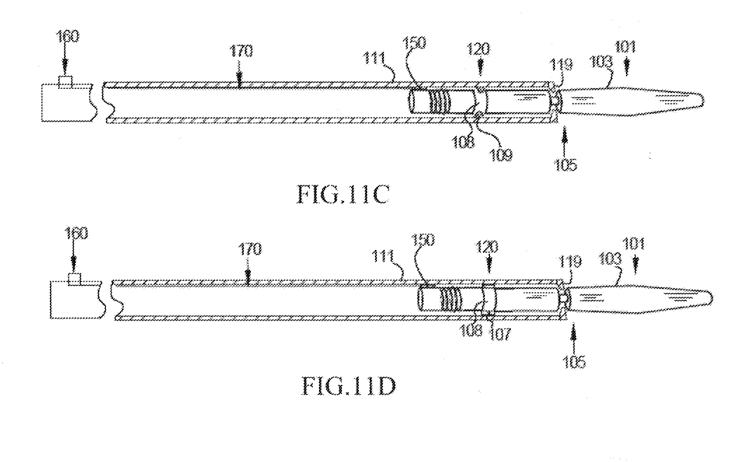

In another embodiment of the present invention, a compression spring may be attached to the guidewire in lieu of a catheter with internal windings, for the purpose of creating a forward propelled drilling guidewire. The guidewires of the present invention, as shown in FIGS. 11A-D, comprise a proximal shaft (111) encasing the proximal half of a distal rotatable body (105). The distal rotatable body 105 comprises two components: the proximal rotatable shaft 120 (which may be encircled by two mechanical securing rings (106, 140) with a compression spring is attached to the proximal ring), and a rotatable tapered head 101, wherein the two components are approximately equivalent in length in the absence of a filtration system. The tapered head 101 may alternatively comprise a cutting tip. The rotatable shaft 120 is encased by the proximal shaft 111, so that rotatable body 105 remains stable without trembling and side movement while spinning. Unlike the previously disclosed occlusion system, the guidewire of the present invention does not possess helical windings on the distal rotatable body, nor are helical windings required on any catheter which may be used with this system.

As previously disclosed in the co-pending application by the inventors, the present invention may comprise various coupling mechanisms between rotatable body 105 and guidewire proximal shaft 111. The guidewire proximal shaft 111 has a narrowing distal end 119, such that it prevents the rotatable shaft 120 from becoming detached from the guidewire proximal shaft 111. In a preferred embodiment shown in FIG. 11A, the rotatable shaft 120 encased by the guidewire proximal shaft 111 is about one third smaller in diameter than the distal part of the rotatable body 105 entering into the catheter 102. Two rings (106, 140) are secured around the rotatable shaft 120, the distal ring for example by clockwise winding, so that a clockwise rotation movement enables further screwing and security clockwise, and the proximal ring by counterclockwise winding, so that a counterclockwise rotation movement enables further screwing and security counterclockwise, such that the rings will not be disconnected from the rotatable shaft 120. Two indented slots 103 emerging from the proximal shaft are disposed between the rings that function to secure the rotatable body 105 to guidewire proximal shaft 111 by preventing movements distally, proximally and laterally, while enabling free rotational movement.

In another embodiment shown in FIG. 11B, an elevated circular slot 107 is disposed close to the rotational shaft 120 between the rings 106 and 140 that function to secure the rotatable body 105 to guidewire proximal shaft 111 by preventing movements distally, proximally and laterally, while enabling free rotational movement.

In two additional embodiments shown in FIGS. 11C & 11D, the mechanical rings 106 and 140 are removed and the compression spring encircles the proximal rotatable shaft 120. In the embodiment exemplified in FIG. 11C, the rotatable shaft 120 has a circular channel 108 around its diameter. The guidewire body may have two indented slots 109 emerging from the guidewire body inner wall and disposed diagonally on the body diameter. The indented slots 109 allow coupling with the circular channel 108 of the guidewire proximal shaft 111, thereby securing the rotatable body 105 to the guidewire proximal shaft 111 and preventing movements distally, proximally and laterally, while enabling free rotational movement.

In another embodiment shown in FIG. 11D, a circular slot 107 emerging from the guidewire body inner wall is coupled with the circular channel 108 of the guidewire proximal shaft 111. This too secures the rotatable body 105 from moving distally or proximally and preventing side leaning within the proximal shaft 111, while enabling free rotational movement.

Proximal Ring with Spring Attached

As shown in FIGS. 11A & 11B the present invention further comprises a rotational shaft 120 further encasing two rings 106 and 140 wherein a compression spring is attached to the proximal ring. The distal ring is solid and functions to secure the distal body 105 into the guidewire proximal shaft 111 at a point about 0.5 mm below the narrowing edge 119 while still permitting rotation of body 105. The proximal ring is encircled by and attached to a coil or helical compression spring, and further comprises a means for releasing the spring from a state of compression. The proximal ring 140 may comprise one or two slots for insertion of a mechanism to hold the spring in a state of compression. Therefore, the proximal ring and spring function as a means to activate axial and rotational movement of the guidewire through a lumen or cavity, as disclosed herein. Likewise, in FIGS. 11C & 11D, the two rings are replaced by a proximal rotatable shaft of a diameter comparable to those of the securing rings, with a coil or helical compression spring encircling the proximal end of the shaft. In all of the embodiments, the spring may be compressed clockwise or counterclockwise. Upon the spring's release, the distal body 105 rotates in the opposite direction of compression with a high velocity (revolutions per minute) and force.

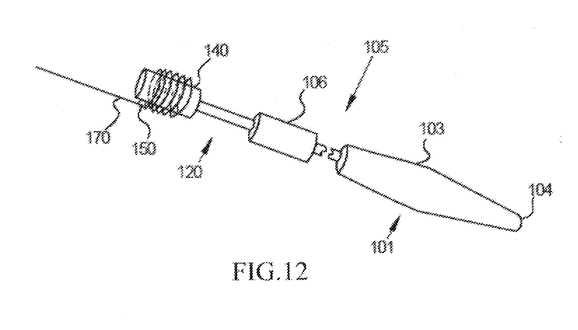

By way of exemplification as shown in FIG. 12, the proximal ring of the present invention comprises one, or two slots (150), wherein a stopper mechanism 170, such as a metal extensor, is inserted into the slot to prevent rotation of the guidewire when the spring is compressed. The extensor 170 is preferably metallic in composition, but may also comprise any non-flexible material. The metal extensor 170 is further attached to one, or preferably two connections, such as wires, that run from the proximal ring to a connection point on the handle of the guidewire. For example, the connection point may be a button 160. When the button is depressed, the extensor is removed from the proximal ring and the guidewire's spring is released from a state of compression, thus causing the guidewire distal body and tip to rotate. By way of exemplification, the spring may rotate upon release three to five times, thus causing the guidewire tip to rotate three to five times at high velocity (revolutions per minute) and axial force so as to pierce through an occlusion.

The spring mechanism of the present invention generates a drilling force in the guidewire tip that is proportional to the magnitude of the force restoring the spring to its resting position in a relaxed, extended state. The spring is rotated clockwise or counterclockwise and compressed to a predetermined position during the manufacturing process, or optionally, by the clinician prior to inserting the guidewire into the body lumen. During the process of compression, the proximal ring 140 and/or proximal rotatable shaft 120 of FIG. 12 are forced to rotate with the spring until locked into position by, for example, inserting a stopper mechanism into the slots of the proximal ring. When the spring is released, by for example removing the stopper, it forces the proximal ring, rotatable shaft and body, and the guidewire tip to rotate at high velocity and force in the opposite direction to which is was compressed (clockwise or counterclockwise). Each spring may only be deployed one time, and the force generated at the rotating guidewire tip is a function of the number of turns the spring is initially compressed. The spring may be the same diameter or slightly wider than the proximal ring while still permitting free rotation of the distal rotatable body within the proximal shaft. One of skill in the art would readily recognize the type and size of spring for use in the present invention (e.g., stiffness, diameter, length, and material composition) and methods of attaching the spring to the proximal ring (e.g., welding).

Safety Control Mechanism: The guidewire of the present invention comprises an inherent safety control system by allowing the clinician to control the amount of force generated by the guidewire as a function of the amount of compression within the device's spring. For example, the guidewire may be set to generate a minimal force by selecting a guidewire with only one rotation of the tip so as to reduce the likelihood of generating excessive forces at the guidewire tip that could penetrate a vessel wall.

Method of Use with Balloon Catheters

The guidewire of the present invention may optionally be used in conjunction with traditional balloon catheters. In a traditional balloon catheter, the clinician inserts the distal end of the guidewire (with its rotating tip) into a body lumen (e.g., artery) and pushes the guidewire proximal end to the site of an occlusion, or point wherein the puncturing of the lumen is of concern. The clinician then places a balloon catheter over the guidewire and pushes it to the guidewire's distal end. (Alternatively, the catheter is inserted into the body cavity first, followed by threading the rotating guidewire through the catheter.) The balloon may then be inflated to center the guidewire rotating tip with the lumen to prevent puncturing or tearing of the lumen walls. Additionally, the balloon catheter may further be used to stabilize the catheter providing back up support to the guidewire, to clear the occlusion and to deliver the stent after successfully wiring.

Rotational Guidewire with Compression Spring and Filtration System

Furthermore, this embodiment of the present invention may further comprise the spring compression system and rotational guidewire combined with the filtration system disclosed supra and shown in FIGS. 6-10. The present invention comprises a filtering system with one-way valves within the hollow guidewire of this embodiment. The filter system functions with the guidewire to simultaneously drill and aspirate debris and then return the filtered blood to the body. Use of the filtering system with the disclosed guidewire allows treatment of all types of occlusions, to include those located within the brain and kidneys because of the filter's ability to prevent life threatening embolizations.

Specific embodiments of the present invention are offered for illustrative purposes only, and are not intended to limit the invention in any manner. Those of skill in the art will readily recognize a variety of non-critical parameters and configurations which may be changed, substituted, or modified to yield essentially the same results and performance while engaging in no more than mere routine experimentation.

What is claimed is:

1. A system for treating an arterial occlusion comprising, in combination:
an outer catheter with a centering balloon and an inflation lumen or tube;
an inner catheter residing within and connected to the outer catheter and possessing an inner lining with spherical threads; and
a drilling guidewire comprising a spherical windings, a proximal shaft and a rotational body; and
wherein the inner catheter spherical threads and the guidewire spherical windings connect when a translational force is applied to the proximal shaft so as to generate a drilling force at the guidewire tip.

2. The system of claim 1, wherein the balloon is inflated to about 0.5 centimeters beyond the inner catheter distal end to secure and center said system without contacting said guidewire.

3. The system of claim 1, wherein said rotational body comprises a proximal rotational shaft and at least one securing ring, and wherein said rotational shaft resides within said proximal shaft.

4. The system of claim 3, wherein said rotational body further comprises a rotational distal body with a tapered end.

5. The system of claim 4, wherein said rotational body further comprises a cutting edge affixed to said tapered end to permit crossing of the occlusion.

6. A method of the system of claim 1 comprising the steps of:
providing an assembly having:
an outer catheter with a centering balloon, and
an inner catheter residing within and connected to the outer catheter;
advancing said assembly through an artery to the proximal cap of said occlusion;
inflating said balloon to secure and center said assembly;
advancing a drilling guidewire through the inner catheter by applying a translational force to the guidewire proximal shaft;
generating a rotational force at the guidewire distal body when the inner catheter spherical threads connect with the guidewire spherical windings; and,
penetrating said proximal cap with said guidewire.

7. The method of claim 6, further comprising affixing a cutting tip to said guidewire and drilling through a calcified occlusion body.

8. The method of claim 7, wherein said guidewire is hollow and houses a filtration system having means for aspirating, filtering occlusion debris, and returning filtered blood to the occlusion site.

9. The method of claim 8, further comprising means for administering therapeutic and diagnostic agents through the delivery tube to the occlusion site.

10. A system for treating an arterial occlusion comprising, in combination:
a guidewire body comprising a proximal shaft encasing part of a distal rotatable body,
said distal rotatable body comprising a compression spring attached to a proximal rotatable shaft and a distal rotatable tapered head; and
wherein said tapered head rotates when said compressed spring is released.

11. The system of claim 10, wherein said proximal rotatable body comprises a distal ring and a proximal ring, and said compression spring is affixed to said proximal ring and to a metal extensor inserted into said proximal ring to prevent rotation of said distal rotatable body.

12. The system of claim 11, wherein said tapered head rotates when said metal extensor is removed from within said proximal ring.

13. The system of claim 10, wherein said compression spring affixes to the proximal end of the rotatable body, and a metal extensor is inserted into a slot within the rotatable body.

14. The system of claim 13, wherein said tapered head rotates when said metal extensor is removed from within said slot.

15. A method for treating an occlusion comprising the steps of:
providing an assembly comprising:
a guidewire body comprising a proximal shaft encasing the proximal end of a distal rotatable body; wherein said distal rotatable body comprises a rotatable shaft housed within the proximal shaft, with two securing rings and a compression spring affixed to said rotatable shaft; and a distal rotatable tapered head;
advancing said assembly through an artery to the proximal cap of said occlusion;
rotating said tapered head by releasing said compression spring; and
penetrating said occlusion with said head.

16. The system of claim 15, wherein said spring is secured in a compressed position by affixing the proximal ring with said stopper mechanism.

17. The method of claim 15, further comprising affixing a cutting tip to said guidewire and drilling through a calcified occlusion body.

18. The method of claim 15, wherein said guidewire is hollow and houses a filtration system having means for aspirating, filtering occlusion debris, and returning filtered blood to the occlusion site.

19. The method of claim 15, further comprising a means for administering therapeutic and diagnostic agents through the delivery tube to the occlusion site.

20. The method of claim 15, further comprising advancing a balloon catheter over said guidewire assembly to the site of an occlusion, and inflating said balloon to secure and center said assembly.
